(19) United States
(12) Patent Application Publication
(10) Pub. No.: US 2024/0180887 A1
(43) Pub. Date: Jun. 6, 2024

(54) USE OF PPAR(gamma) AGONISTS OR MPC INHIBITORS FOR TREATING PRIMARY, INNATE DISEASES OF THE CARDIAC MUSCLE (CARDIOMYOPATHIES)

(71) Applicant: PREVENTAGE THERAPEUTICS GMBH, Starnberg (DE)

(72) Inventor: Cordula WOLF, Starnberg (DE)

(21) Appl. No.: 18/552,575

(22) PCT Filed: Mar. 23, 2022
(86) PCT No.: PCT/EP2022/057708

§ 371 Date: Sep. 26, 2023

(30) Foreign Application Priority Data
Mar. 26, 2021 (EP) 21165271.4

(51) Int. Cl. A61K 31/4439; A61K 45/06; A61P 9/00

(52) U.S. Cl. CPC A61K 31/4439; A61K 45/06; A61P 9/00

(57) ABSTRACT

The present invention relates to PPARγ agonists or MPC inhibitors—in particular, from the group of thiazolidine-2,4-diones—and to a pharmaceutical composition containing suchlike agonists for use in a method for preventing or treating a primary, congenital cardiomyopathy.

All diseases of the cardiac muscle that are genetic are referred to as primary, congenital cardiomyopathies. They first appear in isolated form as diseases of the cardiac muscle (such as hypertrophic cardiomyopathy, dilated cardiomyopathy, arrhythmogenic right ventricular cardiomyopathy, or restrictive cardiomyopathy), or they occur as diseases of the cardiac muscle in the context of a congenital structural heart defect, a genetically-caused syndrome (such as, for example, in the context of a disease from the Noonan syndrome spectrum or other RASopathies), a congenital metabolic disease (such as, for example, in the context of Fabry's disease, mitochondriopathy, or glycogen storage diseases), or of a congenital neuromuscular disease (such as, for example, in the context of Duchenne muscular dystrophy or myotonic dystrophy)¹.

Thus, primary, congenital cardiomyopathies do not arise as a secondary condition in the context of other diseases (such as, for example, in the context of arterial hypertension, diabetes mellitus, a metabolic syndrome, or a coronary heart disease) and are hence independent medical conditions that distinguish themselves from secondary myocardial hypertrophies. These independent medical conditions each require individual therapy concepts. The elimination of such secondary diseases is thus an essential element in the clinical diagnosis of primary, congenital, and in particular hypertrophic, cardiomyopathy^{(2,3,4,5,1)}.

The clinical appearance of primary, congenital cardiomyopathies is characterized in particular by a thickening (hypertrophy) of the cardiac muscle tissue (myocardium) as a result of a dilation of the heart or a combination of both alterations. Also, primary, congenital cardiomyopathies can be accompanied by a fibrosis of the cardiac muscle. In the course of the disease, there is ultimately a reduction in the filling function (diastolic dysfunction) or in the ejection function (systolic dysfunction) of the heart. This involves the clinical symptoms of cardiac insufficiency. Primary, congenital cardiomyopathies are often accompanied by cardiac arrhythmias that are associated with the clinical symptoms of, for example, tachycardias and syncopes, or even sudden cardiac death. The variability of the clinical manifestation thus extends over a wide range, both with regard to clinical symptoms and severity of the symptoms and the time of clinical manifestation. Even newborns can be affected by a severe course; however, the disease can also manifest itself only in early or later adulthood.

Hypertrophic cardiomyopathy (HCM) is defined as an independent condition within the congenital, primary cardiomyopathies. HCM is the by far most common primary, congenital cardiomyopathy. In general, HCM, with a prevalence of about 1:250 to 1:500, is also the most common inherited, genetic heart disease¹. Hypertrophic cardiomyopathy differs pathogenically and pathophysiologically from a thickening of the cardiac muscle, which, as described above, can be observed only as a secondary symptom in other diseases such as, for example, arterial hypertension, heart valve malformation, diabetes mellitus, or metabolic, neurological, or syndrome diseases. Therefore, the clinical diagnosis of HCM requires, inter alia, the exclusion of such secondary diseases^{(2,3,4,5,1)} and is defined as follows according to the guidelines from US and European professional societies:

“ . . . For the purposes of this guideline, we have considered the clinical definition of HCM as a disease state in which morphologic expression is confined solely to the heart. It is characterized predominantly by LVH in the absence of another cardiac, systemic, or metabolic disease capable of producing the magnitude of hypertrophy evident in a given patient and for which a disease-causing sarcomere (or sarcomere-related) variant is identified, or genetic etiology remains unresolved.”⁶

“ . . . The generally accepted definition of HCM is a disease state characterized by unexplained LV hypertrophy associated with nondilated ventricular chambers in the absence of another cardiac or systemic disease that itself would be capable of producing the magnitude of hypertrophy evident in a given patient . . . ” (2011 ACCF/AHA Guideline for the Diagnosis and Treatment of Hypertrophic Cardiomyopathy: Executive Summary⁵)

“ . . . Left ventricular wall thickening is associated with a nondilated and hyperdynamic chamber (often with systolic cavity obliteration) in the absence of another cardiac or systemic disease . . . ” (American College of Cardiology/European Society of Cardiology Clinical Expert Consensus Document on Hypertrophic Cardiomyopathy³)

As already explained above for primary, congenital cardiomyopathies in general, cardiac muscle thickening (hypertrophy) and scarring (fibrosis) of the myocardium are also found to be progressive in HCM with increasing age². Clinically, this results in cardiac dysfunction, cardiac insufficiency, and cardiac arrhythmia, which increase the risk of sudden cardiac death. The genetic cause of HCM are mostly mutations in genes which encode for proteins of the sarcomere (e.g., genes MYBCP3 encoding for MyBP-C, MYH7 encoding for the heavy β-chain of the myosin, TNNI3 encoding for cardiac troponin I, or TNNT2 encoding for cardiac troponin T)^{3,4}. The correspondingly modified structural proteins of the cardiac muscle cell change its biophysical contractility⁵, the cellular energy⁶, and calcium balance⁷, and induce pro-hypertrophic and pro-fibrotic signaling pathways^{8,9,10}. These correlations are summarized in FIG. 1.

However, in principle, a change in the genome of a patient cannot be clearly detected with genetic standard tests when HCM is suspected. Epigenetic factors, among other things, may be responsible for this, or the lack of availability of cardiac muscle tissue for genetic testing. This is because, typically, genetic testing is carried out using other tissue that is easier to obtain, such as, for example, blood or epithelial cells, so that a possible genetic mosaic showing the characteristic mutations of the cardiac muscle tissue cannot be recognized therein. The course of the disease varies greatly, because multiple factors affect the severity of the disease in addition to genetic predisposition^{11,12,13,14,15} (FIG. 1).

In the prior art, pharmacological approaches to treating primary, congenital cardiomyopathies in general and hypertrophic cardiomyopathy in particular are at best, if at all, the subject of preclinical or clinical studies, but have not yet been applied in clinical practice. A positive effect of the calcium antagonist diltiazem^{7,8} and of the angiotensin II receptor antagonist losartan⁹ was provisionally described in animal models. First clinical studies show that diltiazem administration could slow down left ventricular remodeling¹⁰. The clinical results of losartan administration in the prior art are contradictoryl^{11,12}. Furthermore, the inhibition of cardiac myosin ATPase by MYK-461¹³ has been examined in a mouse model in the prior art. A positive effect of this cardiac myosin inhibitor (mavacamten) in patients with obstructive cardiomyopathy has been recently shown in a clinical phase III study¹⁴.

Therefore, due to the lack of availability of effective causal or disease-modifying pharmacological therapy approaches, hypertrophic cardiomyopathy is usually treated exclusively conservatively upon diagnosis, e.g., by avoiding excessive stress on the heart or by refraining from competitive sports, and is controlled at regular intervals. Medical intervention focuses on the prevention of sudden cardiac death, on the symptomatic drug treatment of a possibly occurring cardiac insufficiency with, for example, beta blockers, ACE inhibitors and calcium antagonists, and on the attempt to limit the progression of myocardial remodeling by means of conservative measures^{16,17}. An implantable cardioverter-defibrillator (ICD) is optionally considered in severe cases, which may protect patients from premature death by acute cardiac arrest, but cannot stop the progression of the disease. In the presence of a high-degree constriction of the left ventricular outflow tract by the thickened cardiac muscle, open heart surgery (septal myectomy surgery) or transcoronary ablation of septal hypertrophy (TASH) may be necessary. A heart transplant is a treatment alternative for only very few patients due to lack of availability of donor organs, invasiveness of the surgery and the risks associated therewith, and the necessary lifelong immunosuppression.

In light of all this, the object of the present invention is to provide a pharmacological treatment option for preventing and treating primary, congenital cardiomyopathies—in particular, for preventing and treating hypertrophic cardiomyopathy (HCM).

This object is achieved by the surprising finding that pharmacological intervention in the form of activating the peroxisome proliferator-activated receptor gamma (PPARγ) signaling pathway and/or inhibiting the mitochondrial pyruvate carrier (MPC) offers great preventive or therapeutic potential for the above disease spectrum. Therefore, a PPARγ agonist and/or MPC inhibitor for use in a method for preventing or treating primary, congenital cardiomyopathies is provided according to the invention. Primary, congenital cardiomyopathies that can be preventively or therapeutically treated, in particular, are hypertrophic cardiomyopathy (HCM), dilated cardiomyopathy (DCM), restrictive HCM, as well as cardiomyopathy in the context of a structural, congenital heart defect, diseases from the Noonan syndrome spectrum or other RASopathies, and other congenital system diseases—especially hypertrophic cardiomyopathy (HCM). HCM is usually inherited in an autosomal dominant way; there is thus a 50% chance that an offspring of the affected individual will also have this genetic disorder. In rarer cases, this disease is inherited in an autosomal recessive or matrilineal way. Preferably, the autosomal dominant form of HCM is treated according to the invention.

In the human organism, three PPAR subtypes (α, β/δ, γ) are known, the tissue-specific expression and, in particular, gene expression patterns of which are just as diverse as the biological function of the genes whose transcription is being influenced^{15,16}. PPARγ is expressed ubiquitously^{17,18}.

Hence, the PPARγ agonists or modulators used according to the invention interfere with the signal cascade at the peroxisome proliferator-activated receptor signaling pathway. Peroxisome proliferator-activated receptors (PPAR's) belong to a group of receptors located in the nucleus¹⁹. They can be activated via a physiological or pharmacological ligand and, as transcription factors, can regulate the expression of a plurality of genes. Upon activation, the PPAR's bind to a likewise activated retinoid X receptor (RXR). This complex then binds to a specific DNA sequence, viz., the PPAR response element (PPRE), thereby inducing specific gene transcription patterns^{20,21,22}.

The invention thus discloses a PPARγ agonist or modulator as an active agent for a corresponding prevention or therapy—in particular, an active agent from the drug class of thiazolidinediones, such as, for example, pioglitazone or rosiglitazone—for the prevention and treatment of primary, congenital cardiomyopathies, and in particular for the prevention and treatment of HCM.

The active agent used as the PPARγ agonist, or a glitazone, can be an enantiomer, tautomer, hydrate, solvate, or salt of the respective active agent.

The active ingredient class of thiazolidinediones has been known for decades from a different context. Anti-diabetic effects of thiazolidinediones were described for the first time in the late 1980^{23,24,25}. They increase insulin sensitivity and prevent hyperinsulinemia. Thiazolidinediones are therefore also referred to as insulin sensitizers^{26,27,28,29,30}. For instance, a representative of this class of drugs, rosiglitazone, was used in the treatment of type-2 diabetes mellitus (trade name: Avandia, GlaxoSmithKline); however, due to the observation of cardiovascular side effects accompanied by a significant increase in heart attack risk, it was taken off the market in numerous countries. Overall, it can be said that the data collected and the surveys made in the prior art, insofar as they are concerned with the PPARγ signaling pathway in general and with the therapeutic potential of PPARγ agonists such as thiazolidinediones in particular, relate to cardial diseases which are the secondary result of other underlying conditions, such as, for example, diabetes mellitus, arterial hypertension, the metabolic syndrome, or pulmonary hypertension^{31,32}.

As part of the experimental preclinical studies conducted in connection with the present invention, it has been found, quite surprisingly, that PPARγ agonists and MPC inhibitors, and in particular thiazolidinediones, can have a distinct protective effect on the heart muscle tissue and heart function in the primary, congenital, genetic disease, “hypertrophic cardiomyopathy”—for example, in HOCM (hypertrophic obstructive cardiomyopathy) and HNCM (hypertrophic non-obstructive cardiomyopathy). This observation is diametrically opposed to the prevailing teaching and common medical practice. Although unable to offer a final explanation for these deviating observations, the basis thereof could be a significantly different pathogenesis of primary, genetic cardiomyopathies compared to secondary cardiac muscle diseases. While the diseases addressed according to the invention are to be understood as the result of congenital genetic alterations (for example, of genes encoding for proteins which form sarcomeres of the cardiac muscle tissue, e.g., alterations in the MYBCP3 genes encoding for MyBP-C, MYH7 encoding for the heavy β-chain of the myosin, TNNI3 encoding for cardiac troponin I, or TNNT2 encoding for cardiac troponin T), which congenitally cause the corresponding cardiac muscle alterations and lead to a progressive development, the secondary cardiac muscle diseases are in principle based upon other etiologies which each require other individual therapy approaches.

Cardiomyopathies which are triggered, for example, by inflammatory processes and are therefore not congenital, but acquired, are different conditions than primary, congenital, genetic cardiomyopathies. They, too, must be distinguished etiologically and therapeutically from primary, congenital cardiomyopathy. Dilated cardiomyopathy (DCM) also belongs to this case group. Approximately 70% of the patients affected get DCM secondarily as a result of other diseases, e.g., heart valve defects, arterial hypertension, coronary heart disease, chronic alcohol consumption, or also autoimmune diseases, or as a result of a chronic muscle inflammation. Only about 30% of the patients have familial, i.e., hereditary, DCM. In the prior art, DCM is treated pharmacologically only in accordance with general approaches for the treatment of chronic heart insufficiency (e.g., by means of β-blockers or ACE inhibitors), without taking into account its etiologic causes. A treatment option for the congenital (hereditary) form of DCM therefore does not exist.

In one embodiment of the present invention, the primary, congenital cardiomyopathy does not include the hereditary form of DCM.

In another embodiment, a PPARγ agonist or MPC inhibitor can be used according to the invention, wherein the primary, congenital cardiomyopathy is selected from the group consisting of hypertrophic cardiomyopathy (HCM), a cardiomyopathy in structural, congenital heart defects and in RASopathies—in particular, diseases from the Noonan syndrome spectrum.

In one embodiment, according to the invention, the PPARγ agonist and/or MPC inhibitor used for prevention or therapy is a compound from the group of thiazolidine-2,4-diones. The ring structure of the thiazolidine-2,4-dione is the characteristic backbone of the compounds of this compound class, wherein, typically, a comparatively long-chain substituent is introduced at the C5 ring atom. It is therefore preferably a C5-substituted thiazolidine-2,4-dione. The substituent preferably has two (hetero)aromatic ring systems connected to one another via a linker structure. The ring systems can, for example, be aromatic 6-ring systems, and in particular a pyridyl ring, a naphthyl ring, or a benzyl ring. In one embodiment, a glitazone—in particular, one or more of the following glitazones selected from the group consisting of ciglitazone, balaglitazone, darglitazone, englitazone, netoglitazone, pioglitazone, rivoglitazone, rosiglitazone, GQ-16, and troglitazone—is provided and used according to the invention. In a specific embodiment, pioglitazone ((RS)-5-{p-[2-(5-ethyl-2-pyridyl)ethoxy]benzyl}-2,4-thiazolidinedione) or rosiglitazone ((RS)-5-({4-[2-(methyl-2-pyridinylamino)ethoxy]phenyl}methyl)-2,4-thiazolidinedione) is used in the prevention or treatment of the diseases described above.

When the representatives of the drug class of glitazones are used according to the invention, one should point out not only their PPARγ agonistic effect, but also their quality as inhibitors of the mitochondrial pyruvate carrier (MPC)³⁴. Without being bound by a theory, the mechanism of action of glitazone can thus additionally or alternatively also be based upon its function as MPC inhibitor. In this respect, glitazones used in accordance with the invention are also disclosed herein as MPC inhibitors. Thus, the present invention discloses also MPC inhibitors, and in particular a compound from the group of glitazones, for use in a method for preventing or treating primary, congenital cardiomyopathies. All features disclosed according to the invention for PARγ agonists are thus disclosed according to the invention also for MPC inhibitors, and in particular those from the group of glitazones, and their use in the aforementioned prophylactic or therapeutic method.

Due to chirality at the C5 ring atom, the glitazones act as enantiomers. On the one hand, a racemate of the enantiomers is used according to the invention for the prevention or treatment.

Alternatively, however, one of the two enantiomers can also be used in enriched, and preferably in pure, form. In principle, an enriched/pure form both of the (S)-enantiomer and of the (R)-enantiomer is possible here. The (S)-enantiomer can be preferred when an increased PPARγ agonistic effect is to be ensured. This is because an activation of the peroxisome proliferator-activated receptor gamma (PPARy) is effected almost exclusively by the (S)-enantiomer.

If glitazones are used in their properties as MPC inhibitors, both (S)-enantiomers as well as (R)-enantiomers of thiazolidinediones are administered. According to the invention, however, typically, a pure or enriched (R)-enantiomer is preferred in such cases. This is because the (R)-enantiomer does not activate PPARγ or activates it only slightly, so that the biological effect is achieved quite predominantly by the MPC mode of action. Also, the spectrum of side effects associated with a PPARγ agonistic effect produced in particular by the (S)-enantiomer, such as weight gain, edema, or an increased risk of fractures, can be basically prevented by the administration of the (R)-enantiomer as MPC inhibitor.

Furthermore, according to the invention, a substitution of the hydrogen atom at the chiral center of the thiazolidinediones by deuterium can be provided in particular when enriched or pure forms of the enantiomers are to be used according to the invention. As a result, the kinetics of the racemization reaction upon administration of enriched or pure forms of the two enantiomers can be changed. This is because thiazolidinediones in enriched or pure enantiomer form are kinetically unstable in vivo in aqueous solution and are racemized within a few hours. At a physiological pH of 7.4 and a temperature of 37° C., starting from the originally pure enantiomer, a racemate in a concentration ratio of approximately 2:1 (originally pure enantiomer: other enantiomer) is present after 10 hours; after about 48 hours, the ratio is nearly 1:1³⁵. As a result of deuteration at the chiral center, a relatively stronger biological effect, e.g., as PPARγ agonist ((S)-enantiomer), can be achieved both by the (S)-enantiomer and the (R)-enantiomer³³. In the case of a preferred activity of the glitazone as MPC inhibitor, a deuterated form of the pure or enriched (R)-enantiomer will be preferred, in order to increase the inhibitory effect on the mitochondrial pyruvate carrier. This is because only low or no activation of PPARγ is triggered by the kinetically retarded racemization reaction of a deuterated (R)-enantiomer which, according to the invention, is to be used as an MPC inhibitor.

PPARγ agonists or MPC inhibitors, and in particular glitazones, can be used preferably as salts. Salts of an inorganic or organic acid are suitable salts. In particular, salts of hydrochloric acid or of maleic acid are provided, such as rosiglitazone maleate or pioglitazone hydrochloride.

A pharmaceutical composition is also provided according to the invention, which contains a PPARγ agonist or MPC inhibitor, and optionally a pharmaceutical carrier. It also serves for use in a method for the prevention or treatment of a primary, congenital cardiomyopathy, or is used for the prevention or treatment of a primary, congenital cardiomyopathy—in particular, the aforementioned primary, congenital cardiomyopathies selected from the group consisting of hypertrophic cardiomyopathy (HCM), dilated cardiomyopathy (DCM), restrictive HCM, a cardiomyopathy in structural congenital heart defects, diseases from the Noonan syndrome spectrum, or other RASopathies and other congenital systemic diseases—in particular, hypertrophic cardiomyopathy (HCM). In one embodiment, the pharmaceutical composition is not used for treating the hereditary form of DCM. In another embodiment, a pharmaceutical composition may be employed in accordance with the invention, wherein the primary, congenital cardiomyopathy is selected from the group consisting of hypertrophic cardiomyopathy (HCM), a cardiomyopathy in structural congenital cardiac defects and in RASopathies—in particular, diseases from the Noonan syndrome spectrum.

From a functional perspective, the PPARγ agonists or MPC inhibitors used according to the invention are, in particular, further characterized in that they also inhibit hypoxia-inducible factor 1-alpha.

The pharmaceutical composition to be used according to the invention can contain a racemic mixture of a glitazone, or a pure or enriched (S)-enantiomer or (R)-enantiomer of a glitazone—preferably with a deuterium atom at the chiral center. A racemic mixture as an active ingredient of the pharmaceutical composition can be preferred, for example, when both enantiomers are to be mechanistically effective. In contrast, enriched or enantiomer pure forms are preferred when a specific mode of action based upon the corresponding enantiomer is preferred, and/or the mode of action of the other enantiomer is to be excluded. For example, an (R)-enantiomer may be preferred in the pharmaceutical composition if, for example, the PPARγ agonism is to be avoided, and a biological effect as MPC inhibitor is to be increased. Or else, the (S)-enantiomer in the composition can be present in enriched or pure form if, preferably, a PPARy-based mode of action is intended. In this context, “enriched” means in particular that the one enantiomer fraction makes up at least 70%, and preferably at least 80%, of the total weight amount of the active agent in the pharmaceutical composition.

According to the invention, an (R)-enantiomer of a thiazolidinedione may be preferred in the pharmaceutical composition or as a PPARγ agonist in pure form or, in any case, in enriched form, if it is intended to achieve both significantly lessened hypertrophy and significantly lessened fibrosis of the myocardium (in contrast to hypertrophy or fibrosis without treatment with glitazones). However, if only a modest reduction in hypertrophy (compared with hypertrophy without treatment with glitazones) is intended, it is also possible to use the (S)-enantiomer in enriched or pure form. Depending upon the intended therapeutic outcome, it is also possible to set mixing ratios of (R)-enantiomer and (S)-enantiomer in a pharmaceutical composition according to the invention—for example, mixing ratios in a range of 1:1 to 5:1 or 3:1 to 5:1 ((R)-enantiomer to (S)-enantiomer).

The pharmaceutical composition can be present in liquid, semi-liquid, gel-like, or, in particular, solid form. It can be administered by different routes of administration, as required. Preference is given to oral administration—in particular, in tablet form—for example, as a coated tablet. However, intracardiac or intravenous administration—in particular in the form of an injection or infusion—may also be considered.

The pharmaceutical composition according to the present invention may optionally contain an additive. Examples of the additive may be one or more of excipients, disintegrants, binders, lubricants, colorants, pH adjusters, surfactants, stabilizers, corrective agents, sweeteners, flavorings, fluidizers, antistatic agents, light stabilizers, antioxidants, reducing agents, chelating agents. They—for example, also two or more of these additives—can be mixed and used in a suitable ratio.

Examples of an excipient are crystalline cellulose, anhydrous calcium phosphate, anhydrous double-based calcium phosphate, calcium hydrogen phosphate, precipitated calcium carbonate, calcium silicate, powder cellulose, gelatin, light anhydrous silica (e.g., light anhydrous silica without hydrophobing treatment or amorphous silicon dioxide fine particles having a particle size of more than 0.1 micron), synthetic aluminum silicate, magnesium alumino metasilicate, magnesium oxide, calcium phosphate, calcium carbonate, and calcium sulfate. Crystalline cellulose is preferred, e.g., CEOLUS KG801, KG802, PH101, PH102, PH301, PH302, PH-F20, RC-A591NF (trade names, made by Asahi Kasei Chemicals Corporation), including so-called macrocrystalline cellulose.

Examples of a disintegrant are carboxymethyl cellulose, carboxymethyl cellulose calcium (carmellose calcium), sodium carboxymethyl starch, carmellose sodium, croscarmellose sodium, low-substituted hydroxypropyl cellulose [preferably low-substituted hydroxypropyl cellulose having a content of hydroxypropoxy groups of 5-16 wt %, such as LH-11, LH-21, LH-31, LH-22, LH-32, LH-20, LH-30, LH-33, LH-B1, NBD-020, NBD-021, NBD-022 (trade names, made by Shin-Etsu Chemical Co., Ltd.), and the like. Examples of a binder are hydroxypropyl cellulose—preferably HPC-SSL, SL, L (trade names, NIPPON SODA CO., LTD.)—hydroxypropyl methylcellulose, povidone (polyvinylpyrrolidone), arabic gum powder, gelatin, pullulan, methylcellulose, crystalline cellulose, low-substituted hydroxypropyl cellulose—preferably low-substituted hydroxypropyl cellulose having a content of hydroxypropoxy groups of 5-16 wt %, such as LH-11, LH-21, LH-31, LH-22, LH-32, LH-20, LH-30, LH-33, LH-B1, NBD-020, NBD-021, NBD-022 (trade names, made by Shin-Etsu Chemical Co., Ltd.)—dextran, and polyvinyl alcohol.

Examples of a lubricant are stearic acid, magnesium stearate, calcium stearate, talcum, sucrose esters of fatty acids, sodium stearyl fumarate, waxes, DL leucine, sodium lauryl sulfate, magnesium lauryl sulfate, macrogol, and light anhydrous silica (light anhydrous silica without hydrophobing treatment or amorphous silicon dioxide fine particles having a particle size of more than 0.1 micron). Sodium stearyl fumarate is preferred. Examples of a colorant include food colorants such as Food Color Yellow No. 5 (Sunset Yellow, same as US Food Color Yellow No. 6), Food Color Red No. 2, Food Color Blue No. 2, and the like, food varnish colors, yellow iron oxide (yellow iron oxide pigment), red iron oxide (red iron oxide pigment), black iron oxide (black iron oxide pigment), riboflavin, riboflavin-organic acid ester (e.g., riboflavin butyric acid ester), riboflavin phosphate or alkali metal or alkaline earth metal salt thereof, phenolphthalein, titanium oxide, lycopene, and beta-carotene.

Examples of a pH adjusting agent are citrate, phosphate, carbonate, tartrate, fumarate, acetate and amino acid salt, ascorbic acid, (anhydrous) citric acid, tartaric acid, and malic acid. Examples of a surfactant are sodium lauryl sulfate, polysorbate 80, polyoxyethylene (160) polyoxypropylene (30) glycol, polyoxyethylene (196) polyoxypropylene (67) glycol, and polyoxyethylene hydrogenated castor oil 60. Examples of a stabilizer are sodium ascorbate, tocopherol, tetrasodium edetate, nicotinamide, cyclodextrins; alkaline earth metal salt (e.g., calcium carbonate, calcium hydroxide, magnesium carbonate, magnesium hydroxide, magnesium silicate, magnesium aluminate), and butylhydroxyanisole. Examples of a sweetener are aspartame, acesulfame potassium, thaumatin, saccharin sodium, and dipotassium glycyrrhizinate. Examples of a flavoring substance are menthol, peppermint oil, lemon oil, and vanillin.

Examples of a fluidizing agent are light anhydrous silica (light anhydrous silica without hydrophobing treatment or amorphous silica fine particles having a particle size of more than 0.1 micron) and hydrated silicon dioxide. Here, the light anhydrous silica must contain only hydrated silicon dioxide (SiO₂ nH₂O) (n is an integer) as the main component; specific examples thereof are Sylysia 320 (trade name, Fuji Silysia Chemical Ltd.), AEROSIL 200 (trade name, NIPPON AEROSIL), and the like. Examples of an antistatic agent are talc and light anhydrous silica (light anhydrous silica without hydrophobing treatment or amorphous silica fine particles having a particle size of more than 0.1 micron). Examples of a light-shielding agent are titanium oxide. Examples of an antioxidant are butylhydroxytoluene (BHT), butylhydroxyanisole (BHA), tocopherol, tocopherol esters (e.g., tocopherol acetate), ascorbic acid or its alkali metal or alkaline earth metal salt, lycopene, and beta-carotene. Examples of a reducing agent are cystine and cysteine. Examples of a chelating agent are EDTA or its alkali metal or alkaline earth metal salts.

A formulation of a pharmaceutical composition of the present invention—in particular, in the form of a tablet—can be prepared by customary methods and using the above-mentioned additives. The formulation can be prepared, for example, by mixing the PPARγ agonist or MPC inhibitor or a salt thereof, e.g., the glitazone or a salt thereof (e.g., pioglitazone hydrochloride), and an excipient to be possibly added, by granulating the mixture, drying the granulate, sieving the same, if necessary, mixing a possibly added lubricant (e.g., sodium stearyl fumarate) therewith, and form-pressing the granulate obtained or the mixture. Mixing may be performed with a mixing machine such as a V-mixer, a tumble mixer, and the like. Granulation can take place, for example, using a high-speed mixing granulator, a fluidized bed dryer granulator, and the like. Compression can take place. The tablet can be shaped by punching—for example, with a single punch tableting machine, a rotary-table tableting machine, and the like.

If necessary, a coating can be applied according to a method customary in the technical field of pharmaceutical preparations, and in particular in the case of a retard preparation formulation. In addition, markings or letters for marking, or a separating line for dividing the tablet, can be applied. Examples of the coating base are sugar coating base, water-soluble film coating base, enteric-coated base, retard film coating base, and the like.

Sucrose can be used as a sugar coating base; furthermore, one or more species selected from talc, precipitated calcium carbonate, gelatin, gum arabic, pullulan, carnauba wax, and the like, can be used in combination. Examples of the water-soluble film coating base include cellulose polymers such as hydroxypropyl cellulose, hydroxypropyl methyl cellulose, hydroxyethyl cellulose, methyl hydroxyethyl cellulose, and the like; synthetic polymers such as polyvinyl alcohol, polyvinyl acetal diethylaminoacetate, aminoalkyl methacrylate copolymer E [Eudragit E (trade name)], polyvinylpyrrolidone, and the like; polysaccharides such as pullulan, and the like. Examples of the enteric-coated base include cellulose polymers such as hydroxypropyl methyl cellulose phthalate, hydroxypropyl methyl cellulose acetate succinate, carboxymethyl ethyl cellulose, cellulose acetate phthalate, and the like; acrylic acid polymers such as methacrylic acid copolymer L [Eudragit L (trade name)], methacrylic acid copolymer LD [Eudragit L-30D55 (trade name)], methacrylic acid copolymer S [Eudragit S (trade name)], and the like; naturally occurring substances such as shellac.

Examples of retard film-coating base include cellulose polymers such as ethyl cellulose, acetyl cellulose, and the like; acrylic acid polymers such as aminoalkyl methacrylate copolymer RS [Eudragit RS (trade name)], ethyl acrylate methyl methacrylate copolymer suspension [Eudragit NE (trade name)], and the like.

In addition, a coating additive for coating can be used. Examples of the coating additive are light stabilizers and/or colorants such as titanium oxide, talcum, red iron oxide, and the like; plasticizers such as polyethylene glycol, triethyl citrate, castor oil, polysorbates, and the like.

In one embodiment, the pharmaceutical composition containing the PPARγ agonist or MPC inhibitor according to the present invention may be formulated for immediate release or, in another embodiment, for delayed release.

When embodied as a retard formulation with delayed release of the active agent, such a retard formulation typically has a release of 25-58% after two hours and 60-100% after 4 h. The release can be determined according to the Paddle method (US Pharmacopeia) in a dissolution test system with 50 rpm (KCL/HCl buffer (pH=2) at 37° C. as a test solution). The Cₘₐₓ value—in particular, of a tablet after oral administration—of a retard formulation with 1 mg of the active agent is preferably within 10-90%, and preferably between 30 and 80%, compared to an immediate release formulation. The Cₘₐₓ value is thus preferably below the peak concentration of an immediate release formulation. Under these conditions, the target AUC value is also within a range of 50 to 150% or 60 to 140% compared to an immediate release formulation.

The formulation can be provided as a retard preparation by using a film coating with a retard film-coating base, as described above. Alternatively, the retard formulation may contain a gel-forming polymer—in particular, selected from polyethylene oxide (e.g., having a molecular weight of 100,000-10,000,000, and preferably 300,000-8,000,000), hypromellose (e.g., with a molecular weight of 20,000-500,000, and preferably 20,000-250,000), hydroxypropyl cellulose, methylcellulose, carboxymethyl cellulose, Na-croscarmellose, and mixtures of the aforementioned polymers. The proportion of the polymer component in the retard formulation can be between 10 and 90%, and preferably between 30% and 80% or 50% to 80%. The release rate of the retard preparation can be controlled by altering the amount and molecular weight of the gel-forming polymer, such as polyethylene oxide, and the like.

The retard formulation can also contain, for example, a gel-forming promoter, which supports the penetration of water before the gel-forming polymer starts to swell. It can, for example, be a water-soluble hydrophilic compound, such as, for example, lactose, glucose, mannitol, trehalose, d-sorbitol, xylitol, sucrose, maltose, lactulose, d-fructose, dextran, glucose, or water-soluble polymers such as polyethylene glycol. It is also possible to use a water-insoluble hydrophilic compound—for example, starch, hydroxypropyl starch, crospovidone, crystalline cellulose, etc.

In case of production of such a retard formulation with a gel-forming polymer, in order to provide an oral formulation, and in particular a tablet, of the pharmaceutical composition according to the invention, a gel-forming polymer (e.g., polyethylene oxide) and optionally a gel-forming promoter (e.g., d-mannitol, lactose) can be added to the mixture to be granulated, as described above. The retard preparation containing a gel-forming polymer can furthermore show rapid gelling, since its gel-forming function is improved by encapsulating one component that prevents the penetration of water into the granulate (hydroxy polymer, binder, and the like), and the path of water penetration is secured by coating the component, which prevents water penetration, with a surface modifier. This enables a zero-order active agent release. Moreover, the strength required to withstand physical stimuli in the body by food intake can be imparted.

The weight of a tablet for oral administration is typically 40-600 mg, preferably 60-480 mg, more preferably 60-200 mg, and even more preferably 100-200 mg.

The PPARγ agonist or MPC inhibitor used according to the invention, and in particular the glitazone used according to the invention, can be administered to children and adults. According to the invention, this enables in particular a pediatric therapy, i.e., for example, of children from 1 to 12 years of age.

The PPARγ agonist or MPC inhibitor—in particular, a glitazone as the active agent of a pharmaceutical composition—can typically be administered at a daily dose of 1 to 30 mg, and in particular 5 to 15 mg, depending upon the sex, age, body weight, form of the primary, congenital cardiomyopathy, progression, therapy duration, and severity, and the selection of the PPARγ agonist or MPC inhibitor—in particular, the glitazone. Administration can be once per day or multiple times per day. When treating children, a formulation of the active agent other than in tablet form may be necessary. Liquid formulations or capsule formulations may be preferred for pediatric treatment.

The PPARγ agonists or MPC inhibitors used according to the invention, and in particular the glitazones used according to the invention or a pharmaceutical composition containing the same, can also be used in a combination therapy. Thus, for example, two or more active agents of the active agent class of glitazones can be combined with one another, i.e., for example, rosiglitazone and pioglitazone. Further combination therapies may include one or more PPARγ agonist(s) or MPC inhibitor(s) used according to the invention—in particular, one or more glitazone(s) or one or more pharmaceutical composition(s) containing the same—in combination with at least one standard therapy according to prior art, which are applied for symptomatic treatment. This standard therapy used in combination can be provided by administering an active agent from the group selected from a beta blocker, an angiotensin-converting enzyme (ACE) inhibitor, an angiotensin I receptor blocker, an aldosterone antagonist, a calcium antagonist, an anti-arrhythmic drug, and a myosin modulator—in particular, for reducing the myosin actin cross-bridge connections—preferably mavacamten (6-[[(1S)-1-phenylethyl]amino]-3-propan-2-yl-1H-pyrimidine-2,4-dione).

The use of one or more of the PPARγ agonists or MPC inhibitors disclosed according to the invention is also disclosed—in particular, of a compound from the group of glitazones—for preparing a drug, e.g., a pharmaceutical composition as herein disclosed according to the invention, for the prevention or treatment of the herein disclosed diseases from the group of primary, congenital cardiomyopathies.

Finally, methods for the prevention or treatment of diseases from the group of primary, congenital cardiomyopathies are disclosed according to the invention, wherein a pharmaceutically effective amount of one or more PPARγ agonists or MPC inhibitors—in particular, an effective amount of one or more thiazolidine-2,4-dione(s) (glitazone(s)) or of a pharmaceutical composition containing the same, as herein disclosed according to the invention—is administered to a subject.

### FIGURES

The figures show the following:

FIG. 1 Pathogenesis of hypertrophic cardiomyopathy (FIG. 1a: describes sarcomere structures of the cardiac muscle; FIG. 1b: describes myocardial diseases as well as mechanistic causes and factors that can lead to myocardial diseases).

FIG. 2 Determination of left ventricular fraction shortening ([(left ventricular end-diastolic diameter minus left ventricular end-systolic diameter) divided by left ventricular end-diastolic diameter]×100) in percent by means of echocardiography as a correlate for myocardial hypercontractility: The development of hyperdynamic left ventricular contractility observed in untreated HCM mice (αMHC^{719/+}) 6 weeks after onset of the experiment compared to wild-type (WT) mice (p=not significant) is significantly inhibited by oral treatment with rosiglitazone (Rosi) (p=0.046). The αMHC^{719/+} mice treated with rosiglitazone (5 mg/kg of body weight/day orally) showed a reduced left ventricular fraction shortening compared to untreated αMHC^{719/+} mice.

FIG. 3 Determination of the maximum thickness of the wall of the left ventricle of the heart by means of echocardiography as a correlate for myocardial hypertrophy: The development of strong myocardial hypertrophy observed in untreated HCM mice (αMHC^{719/+}) 6 weeks after onset of the experiment compared to wild-type (WT) mice (p=0.032) is significantly inhibited by oral treatment with rosiglitazone (Rosi) (p=0.046). The αMHC^{719/+} mice treated with rosiglitazone (5 mg/kg of body weight/day orally) showed a reduced left ventricular thickness compared to untreated αMHC^{719/+} mice.

FIG. 4 Presentation of the heart-to-body weight ratio (relative cardiac muscle mass ex vivo after organ removal) as a correlate for myocardial hypertrophy: The development of strong myocardial hypertrophy observed in untreated HCM mice (αMHC^{719/+}) 6 weeks after onset of the experiment (p=0.008) compared to wild-type (WT) mice is completely inhibited by treatment with pioglitazone (Pio) (p=0.008). The ex vivo heart-to-body weight ratio in αMHC^{719/+} mice treated with pioglitazone (10 mg/kg of body weight/day orally) is significantly reduced compared to untreated αMHC^{719/+} mice.

FIG. 5 Examination of the percentages of left ventricular cardiac myosins in the DRX (disordered relaxation) compared to the SRX (super-relaxed) state using a fluorescent ATP analog. Compared to untreated αMHC^{719/+} mice, the treatment with pioglitazone results in a significant shift towards the SRX state, similar to wild-type mice. The treatment of the HCM mouse model with pioglitazone thus also has a positive effect on the functional properties of the left ventricular heart muscle tissue. The results of this Mant-ATP assay indicate that it was possible to prevent the changes in the biophysical properties of the myosins, which are described as being the cause of hypertrophic cardiomyopathy, in the mice treated with pioglitazone. While the untreated αMHC^{719/+} mice showed traits of the typical biophysical changes (significant increase in myosins in the “disordered relaxation” (DRX) state), the treatment with pioglitazone led to a normalization of these abnormalities.

FIG. 6 Histopathology of the left ventricular myocardium of wild-type (WT) and HCM mice (αMHC^{719/+}) with and without treatment with pioglitazone (PIO) after 6 weeks; myocardial fibrosis appears in medium gray in the Masson's trichrome-stained histological sections. Wild-type mice have hardly any fibrosis (WT without (A) and WT with (B) treatment with pioglitazone). HCM mice show massive myocardial fibrosis (white arrows in (C)). The treatment with pioglitazone almost completely prevents the formation of myocardial fibrosis (D). (E) After semi-automated threshold detection of collagen in Masson's trichrome-stained sections in the ImageJ Tool, there is significantly less fibrosis in the HCM mice treated with pioglitazone compared to the untreated HCM mice (p=0.017). Hence, the histopathological examinations overall show significantly less fibrosis in the left ventricular myocardium of αMHC^{719/+} mice treated with pioglitazone (10 mg/kg of body weight/day orally) compared to untreated αMHC^{719/+} mice.

FIG. 7 Relative expression of PPARγ mRNA in the left ventricular myocardium of untreated HCM mice and HCM mice (αMHC^{719/+}) treated with N-acetylcysteine (NAC) or rosiglitazone (Rosi) compared to wild-type mice (WT) after 6 weeks; ***: p<0.001. As regards the effect of PPARγ agonists on the PPARγ mRNA expression in the heart muscle, it can be said that, compared to wild-type mice, there is a significantly reduced mRNA expression of PPARγ in the myocardium of the αMHC^{719/+} mouse. Oral administration of thiazolidinediones (rosiglitazone) increases the myocardial mRNA expression of PPARγ again almost completely to wild-type level.

FIG. 8 The mRNA expression of pro-hypertrophic, pro-fibrotic, and pro-inflammatory genes such as Acta1, Col1a1, Col1a2, Cyba, Gdf15, Hif1a, Icam1, IL33, Mmp2, Nfkb1, Nox4, Nppa, Postn, Tgfb1, Tgfb2, and Tgfb3 is increased in the left ventricular myocardium of αMHC^{719/+} HCM mice (HET) compared to wild-type mice (WT) (HET/WT). By treating the HCM mice with pioglitazone (PIO), overexpression of these proteins which are involved in pro-hypertrophic, pro-fibrotic, and pro-inflammatory signaling pathways and the activation of which is described in the pathological remodeling of cardiac muscle, is significantly reduced or completely prevented (HET+PIO/HET). *:p<0.001.

FIG. 9 The formation of radical oxygen species (ROS) as an expression of oxidative stress, determined by electron paramagnetic resonance spectroscopy (EPR), is significantly increased in the left ventricular myocardium of HCM mice (αMHC^{719/+}) compared to wild-type mice (WT) 6 weeks after onset of the experiment. The production of radical oxygen species in the left ventricular myocardium of HCM mice treated with poglitazone is significantly reduced compared to untreated HCM mice (p=0.053) and comparable to the values measured in wild-type mice. Reduction of oxidative stress: The treatment with PPARγ agonists leads to reduced expression of hypoxia-inducible factor 1-alpha, a major transcription factor in the redox signaling pathway. As a result, less radical oxygen species are produced in the left ventricular myocardium, thereby reducing cellular oxidative stress. A protective effect on the pathological remodeling of the cardiac muscle was observed.

FIG. 10 Design of experiments/experiment record

FIG. 11 Design of experiments/experiment record according to experimental setup C.

FIG. 12 Determination of the maximum thickness of the wall of the left ventricle of the heart by means of echocardiography as a correlate for myocardial hypertrophy: The development of strong myocardial hypertrophy observed in untreated HCM mice (αMHC^{719/+}) 6 weeks after onset of the experiment compared to wild-type (WT vs. αMHC^{719/+}: p<0.001) is significantly inhibited by oral treatment with the (R)-stereoisomer of pioglitazone (10 mg/kg/day) (αMHC^{719/+} vs. αMHC^{719/+} plus (R)-Pio: p<0.001). The effect is less pronounced in αMHC^{719/+} mice treated with the (S)-stereoisomer of pioglitazone (10 mg/kg/day) (αMHC^{719/+} vs. αMHC^{719/+} plus (S)-Pio: p=0.072).

FIG. 13 Determination of myocardial fibrosis using histopathological sections of myocardium. The manifestation of myocardial fibrosis (blue-colored in the Masson's trichrome-stained histological sections) was determined semi-quantitatively by two blinded examiners in each case independently of one another. The scores assigned were 0 to 3:0: no fibrosis, 1: mild fibrosis; 2: moderate fibrosis, 3: severe fibrosis; 0.5: no to mild fibrosis, 1.5: mild to moderate fibrosis; 2.5: moderate to severe fibrosis, 3: severe fibrosis.

The development of strong myocardial fibrosis observed in untreated HCM mice (αMHC^{719/+}) 6 weeks after onset of the experiment compared to wild-type mice (WT vs. αMHC^{719/+}: p<0.001) is significantly inhibited by oral treatment with the R-stereoisomer of pioglitazone (10 mg/kg/day) (αMHC^{719/+} vs. αMHC^{719/+} plus (R)-Pio: p=0.01). The effect cannot be seen in αMHC^{719/+} mice treated with the S-stereoisomer of pioglitazone (10 mg/kg/day) (αMHC^{719/+} vs. αMHC^{719/+} plus (S)-Pio: p=0.916).

FIG. 14 Exemplary representation of histopathological sections of the left ventricular myocardium of a wild-type (WT) mouse and of three HCM mice (αMHC^{719/+}). Of the three HCM mice, one was untreated (αMHC^{719/+}), one was treated with the R-stereoisomer of pioglitazone (10 mg/kg/day) (αMHC^{719/+} (R)-Pio), and one was treated with the S-stereoisomer of pioglitazone (10 mg/kg/day) (αMHC^{719/+} (S)-Pio).

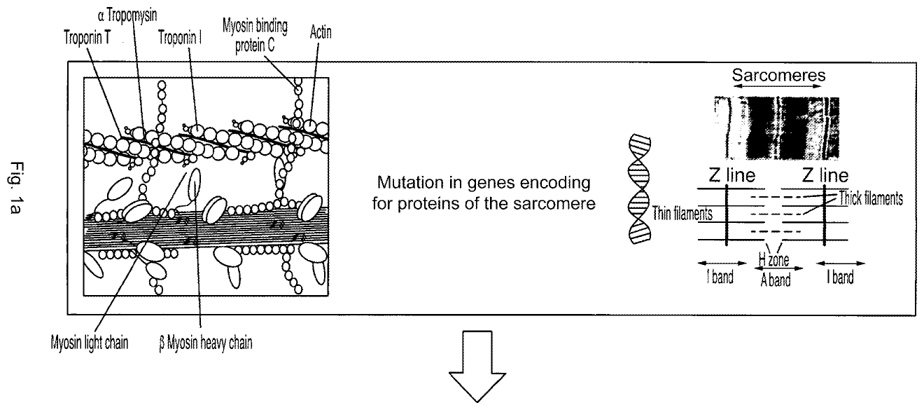

### EXEMPLARY EMBODIMENTS

Preclinical Studies on the Treatment of HCM with Thiazolidinediones (Experimental Setup A/B) or with the Respective Stereoisomers of a Thiazolidinedione (Experimental Setup C/D)

### A) Experimental Setup

Wild-type mice and genetically modified mice were treated and compared with untreated mice. The examination of genetically modified mouse strains allows assessment of the relevance of a certain gene for the pathophysiological phenomena. In the case of hypertrophic cardiomyopathy caused by sarcomere mutations, progressive myocardial hypertrophy, impaired diastolic and systolic ventricular function, structural remodeling of the myocardium with chaotic arrangement of the myocytes (“myocyte disarray”), as well as fibrosis and arrhythmia occur in the hearts of the patients concerned. Since the phenotype of certain genetically generated mouse models for hypertrophic cardiomyopathy is very similar to the phenotypes of the patients concerned, these models are suitable for a holistic examination of the disease^{36,37}.

The examinations were carried out on an established HCM mouse model, in which an arginine-to-tryptophan mutation at position 719 of the heavy chain of the cardiac mouse alpha-myosin gene was effected by homologous recombination (referred to as αMHC^{719/+})³⁸. Such genetically modified mice increasingly develop the typical pathognomonic disease characteristics of hypertrophic cardiomyopathy such as myocardial hypertrophy and fibrosis with increasing age. The phenotype of this genetically generated HCM mouse model thus simulates the phenotype of HCM patients affected^{36,37,38}. The manifestation of the disease can be accelerated by subcutaneous administration of the calcineurin inhibitor cyclosporin A (CsA)⁹.

The animals were genotyped in their 3^{rd} postnatal week.

Between their 6^{th} and 12^{th} postnatal week, all mice were injected subcutaneously with cyclosporin A (CsA) to accelerate hypertrophic remodeling in the myocardium in the HCM mouse model⁹. Half of the mice were treated with the PPARγ agonists rosiglitazone and pioglitazone from the group of thiazolidinediones between their 6^{th} and 12^{th} postnatal week. In their 12^{th} postnatal week, they were subjected to in vivo transthoracic echocardiography. The animals were then euthanized for ex vivo examination (organ removal, histopathology, and determination of radical oxygen species, of mRNA and protein expression in the tissue) (see FIG. 10).D

Only male mice were used for the experiments, because the male animals of the hypertrophic cardiomyopathy mouse line (αMHC^{719/+}) have a more pronounced disease phenotype compared to the female animals^{39,37,38} The group size was 11 animals; at least 6 animals per group were compared with one another for analysis purposes.

In vivo phenotyping of the left ventricular wall thickness and ventricular function was done by means of transthoracic echocardiography, as described above^{40,41,39}.

Examination by echocardiography is done as follows, specifically: removal of the mice from the cage, weighing and visual assessment of the animals, slight sedation with intraperitoneal midazolam after 30 minutes of observation, followed by echocardiography according to standardized, published standard protocols^{39,41,37,40}.

The mice were weighed and euthanized to calculate the heart-to-body weight ratio. The thorax was opened quickly, and the heart was removed and washed twice in cold PBS solution. The large vessels and connective tissue were removed, the heart was swapped dry and weighed, and the heart and body weight of the mouse were determined.

In order to quantify myocardial fibrosis, the removed hearts were fixed in 4% paraformaldehyde, dehydrated, and placed in paraffin, as described above^{37,42,43}. 5-μm sections were stained with Masson's trichrome and hematoxylin-eosin. The extent of myocardial fibrosis was quantified by a digitized method by calculating the percentage of cardiac muscle portions stained blue with Masson's trichrome in relation to the overall stained cardiac muscle. Perivascular and intramurine structures, the endocardium, or trabeculas were excluded in the calculation of the percentage of fibrosis.

Cardiac myosin ATPase activity was measured by Mant-ATP assay, which examines cardiomyocyte contractility and relaxation indirectly by measuring the activity of cardiac myosin ATPase. For this purpose, the Mant-ATP assay (Roche) was applied in the left ventricular myocardium of the different mouse groups, which was shock-frosted in liquid nitrogen and stored in frozen state at −80° C. Mant-ATP is a fluorescent, non-hydrolyzable ATP that binds the head of the cardiac myosin. The assay measures the release of Mant-ATP after addition of ATP^{44,45}.

Electron paramagnetic resonance (EPR) was applied as described in order to calculate the production of radical oxygen species (ROS) in the left ventricular myocardium⁴⁶⁻⁴⁸. In summary, left ventricular myocardium was washed twice in Krebs-HEPES buffer (pH 7.35; 99 mM NaCl, 4.69 mM KCl, 25 mM NaHCO₃, 1.03 mM KH₂PO₄, 5.6 mM D-glucose, 20 mM Na-HEPES, 2.5 mM CaCl₂), 1.2 mM MgSO₄) and then incubated on ice for 20 minutes in Krebs-HEPES buffer supplemented with 25 μM desferoxamine, 5 μM diethyldithiocarbamate, and 100 μM spintrap 1-hydroxy-3-methoxycarbonyl-2, 2,5,5-tetramethylpyrrolidine (CMH; Noxygen). The analysis of ROS generation was carried out at 37° C. for 10 minutes with 20 scans in an Escan EPR spectrometer under temperature control (Noxygen) and the following parameters: microwave power 23.89 mW; center field 3459-3466 G; steep width 10 G, frequency 9.7690 GHz and modulation amplitude 2.93 G. ROS generation was calculated by means of linear regression and normalized to the cellular protein content.

Statistical analysis was performed by means of SPSS (Statistical Package for Social Sciences, IBM, Chicago, IL, version 22). The data are shown in box plots as median, quartiles, minimum and maximum values. Medians of the different groups were checked using the Mann-Whitney test (Wilcoxon test, non-parametric data).

RNA isolation was carried out by means of the peqGOLD Total RNA Kit (Peqlab) from the left ventricular myocardium according to the manufacturer's instructions. First-strand cDNA was synthesized from 500 ng of total RNA, which is reverse-transcribed (Reverse Transcription Kit, Invitrogen) with random hexamers (TIB Molbiol). RtPCR analysis is carried out with Perfecta SYBR Green fast Mix (VWR, Germany) in the QIAGEN Rotor-Gene Q (Corbett Rotor-Gene 6000). Quantification is done by means of ACT calculation and randomization test^{49,50}.

Strand-specific, polyA-enriched RNA sequencing was carried out as described above⁵¹. The RNA was isolated using the AllPrep RNA Kit (Qiagen). The RNA integrity number (RIN) was determined using the Agilent 2100 BioAnalyzer (RNA 6000 Nano Kit, Agilent). In order to prepare the library, 1 μg RNA was poly(A)-selected, fragmented, and reverse-transcribed with the Elute, Prime, Fragment Mix (Illumina). A-tailing, adaptor ligation, and library enrichment were carried out as described in the TruSeq Stranded mRNA Sample Prep Guide (Illumina). RNA libraries were tested for quality and quantity using the Agilent 2100 BioAnalyzer and the Quant-iT PicoGreen dsDNA Assay Kit (Life Technologies). The RNA libraries were sequenced as 100 bp paired-end runs on an Illumina HiSeq4000 platform. The STAR Aligner (version 2.4.2a)⁵² with modified parameter settings (--twopassMode=Basic) was used for the split-read alignment against the human genome assembly hg19 (GRCh37) and the UCSC knownGene annotation. HTseq-count (version 0.6.0)⁵³ was used to quantify the number of reads mapped to annotated genes. FPKM values (fragments per kilobase of transcript per million fragments mapped) were calculated using custom scripts. Differential expression analysis was performed with the R Bioconductor package DESeq2⁵⁴.

### B) Summary of Results

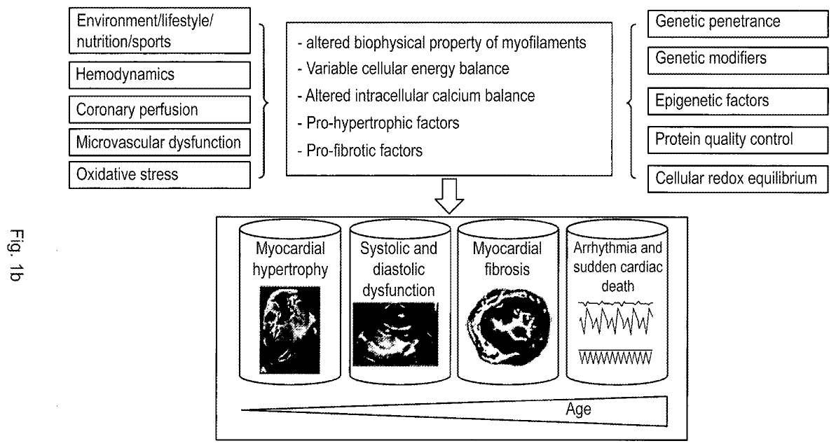

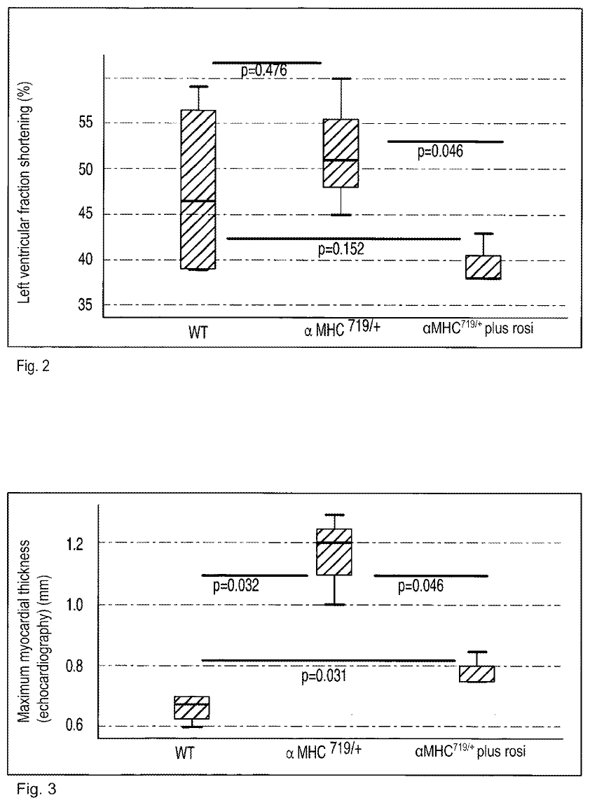

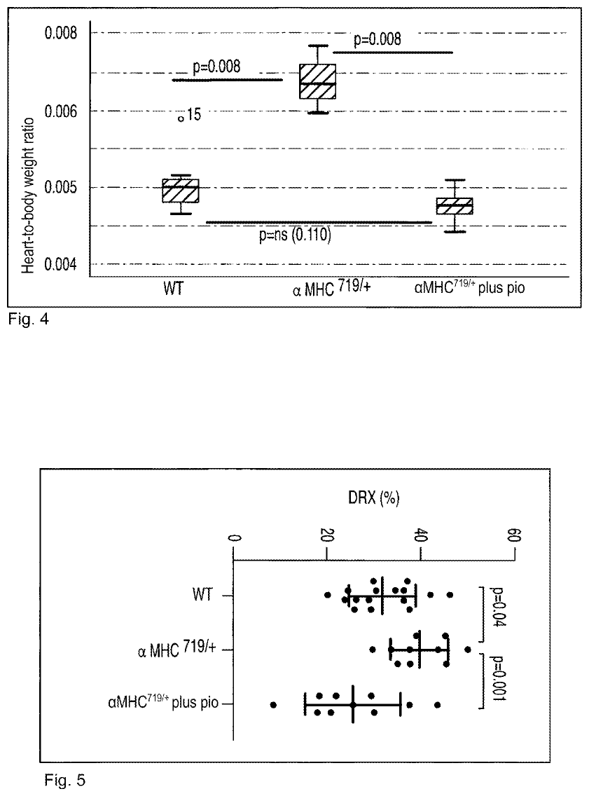

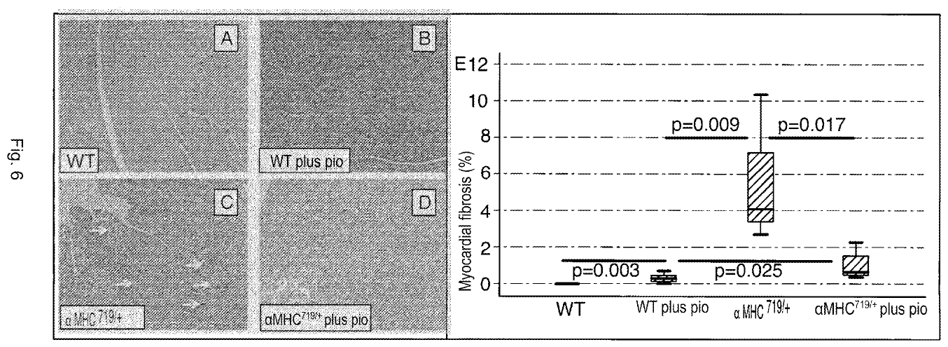

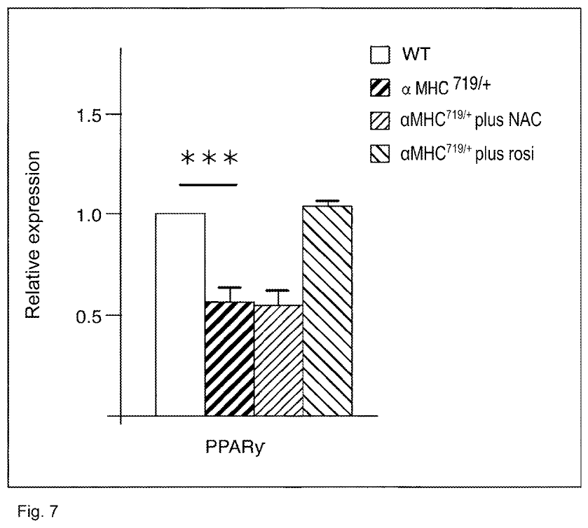

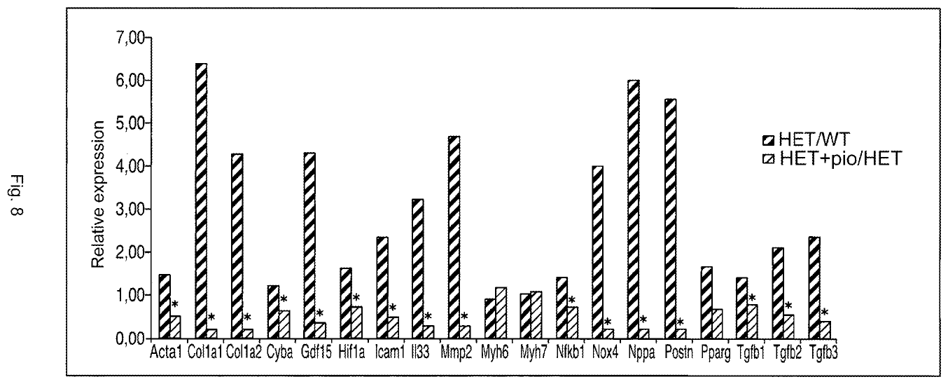

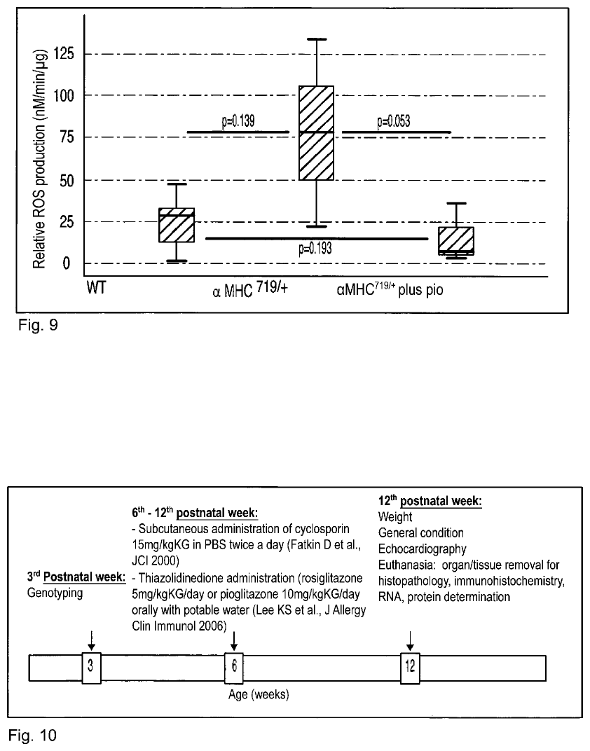

In summary, the results obtained with the preclinical αMHC^{719/+} HCM mouse model show—in analogy to the most frequent genetic mutation in HCM patients—that, compared to wild-type mice, a downregulation of PPARγ in the left ventricular myocardium occurs in the αMHC^{719/+} mouse model and that the oral administration of the PPARγ agonists rosiglitazone and pioglitazone largely prevents, on the one hand, changes in biophysical properties in the heart muscle cell and, on the other, the onset of myocardial hypertrophy and fibrosis in the HCM mouse model. Hypercontractility as determined by echocardiography, and the strong increase in the thickness of the left ventricle in HCM mice compared to wild-type mice are significantly reduced when treated with the PPARγ agonist rosiglitazone. The treatment with pioglitazone also prevents the functional biophysical changes described in the left ventricular myocardium of the HCM mouse model and the overexpression of pro-hypertrophic, pro-fibrotic, and pro-inflammatory genes. It is assumed that at least some of these protective effects are due, inter alia, to the activation of hypoxia-inducible factor alpha (HIF1α) being inhibited⁵⁵. As a result, the redox system is inhibited, and myocardial oxidative stress is reduced. This hypothesis is also supported by the significantly decreased radical oxygen species measured in the left ventricular myocardium of HCM mice treated with pioglitazone. The preclinical HCM mouse model has thus shown that drug treatment with a PPARγ agonist such as, for example, the thiazolidinedione pioglitazone or rosiglitazone, can significantly or almost completely prevent pathological remodeling of the heart muscle in hypertrophic cardiomyopathy. The results of these experiments are shown in FIGS. 2 through 9.

### C) Experimental Setup

This experimental protocol examined the action of the two stereoisomers of a thiazolidinedione (pioglitazone) ((S)- and (R)-stereoisomers) in the same hypertrophic cardiomyopathy mouse model as described for experimental setup A. The experimental setup is thus similar to the experimental setup according to A.

As shown in the experiments in the experimental setup under A), wild-type mice and genetically modified mice were treated and compared with untreated mice. The examination of genetically modified mouse strains allows assessment of the relevance of a certain gene for the pathophysiological phenomena. As described according to experimental setup A, only male mice were used for the experiments, because the male animals of the hypertrophic cardiomyopathy mouse line (αMHC^{719/+}) have a more pronounced disease phenotype compared to the female animals^{39,37,38}. Hence, reference can be made to the above description of the experimental setup according to A).

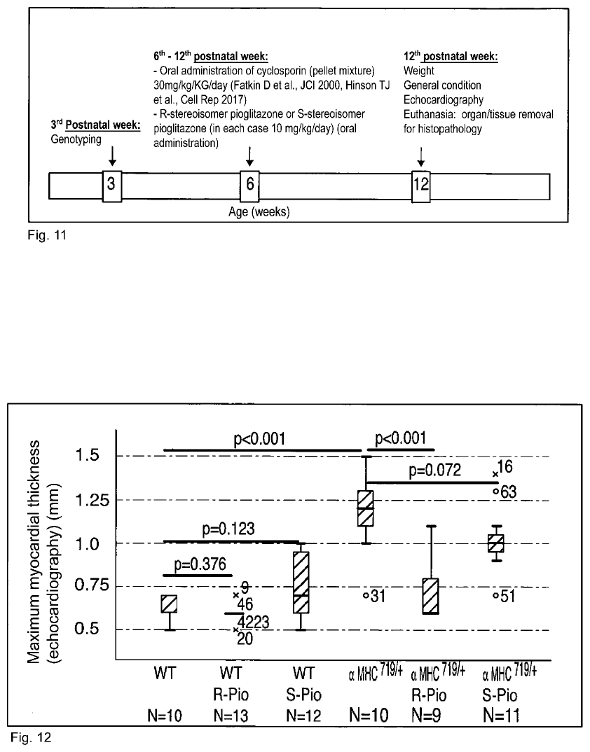

The animals were genotyped in their 3^{rd} postnatal week (as also described in accordance with experimental setup A). Between their 6^{th} and 12^{th} postnatal week, all 65 mice were orally administered a daily dose of 30 mg/kg cyclosporin A (CsA) which was added to their pellet diet to accelerate hypertrophic remodeling in the myocardium in the HCM mouse model⁵⁶. Between their 6^{th} and 12^{th} postnatal week, (a) 13 WT and 9 MHC^{719/+} mice were in addition treated orally with the (R)-stereoisomer of a thiazolidinedione (pioglitazone) (10 mg/kg/day) or (b) 12 WT and 11 αMHC^{719/+} mice were in addition treated orally with the (S)-stereoisomer (pioglitazone) (10 mg/kg/day). In their 12^{th} postnatal week, they were subjected to in vivo transthoracic echocardiography. The animals were then euthanized for ex vivo examination (organ removal and histopathology) (see FIG. 11).

As shown in experimental setup A, in vivo phenotyping of the left ventricular wall thickness and ventricular function was done by means of transthoracic echocardiography, as described above^{40,41,39}.

Examination by echocardiography was done as follows—specifically: removal of the mice from the cage, weighing and visual assessment of the animals, performance of echocardiography according to standardized, published standard protocols in conscious animals after 30 minutes of observation^{39,41,37,40}. In order to quantify fibrosis, the removed hearts were fixed in 4% of paraformaldehyde, dehydrated, and placed in paraffin, as described above^{37,42,43}. 5-μm sections were stained with Masson's trichrome. Semi-quantification of myocardial fibrosis was done independently of one another by two blinded examiners. For this purpose, the percentage of cardiac muscle portions stained blue with Masson's trichrome in relation to the overall stained cardiac muscle was calculated. Perivascular and intramurine structures, the endocardium, and trabeculas were excluded in the calculation of the percentage of fibrosis. The classification was “no fibrosis,” “mild fibrosis,” “moderate fibrosis,” and “severe fibrosis.”

As described according to experimental setup A, statistical analysis was performed by means of SPSS (Statistical Package for Social Sciences, IBM, Chicago, IL, version 22). The data are shown in box plots as median, quartiles, minimum and maximum values. Medians of the different groups were checked using the Mann-Whitney test (Wilcoxon test, non-parametric data).

### D) Summary of Results

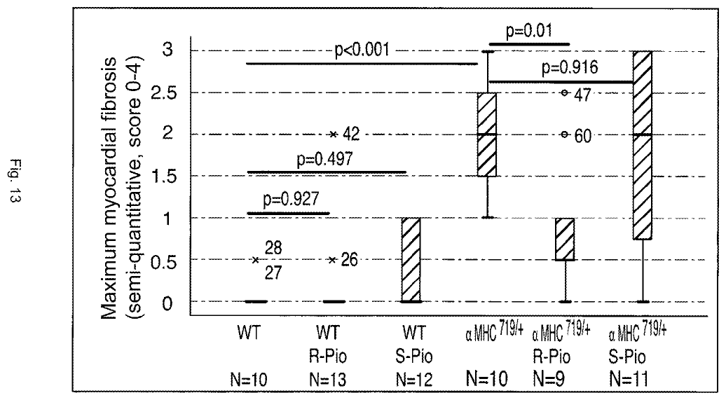

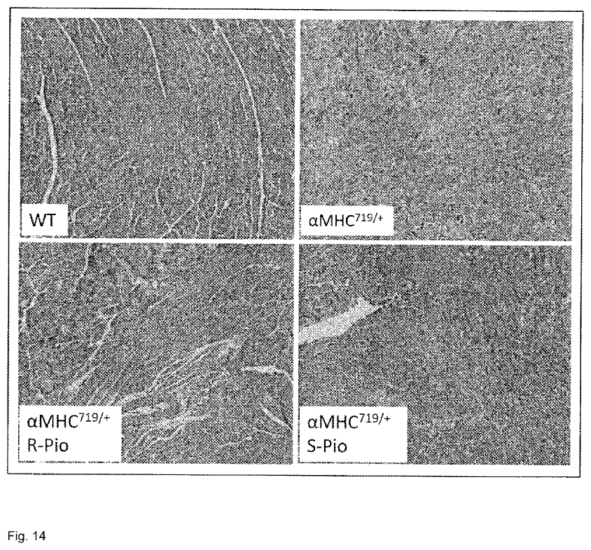

The results of the experiments according to experimental setup C) are shown in FIGS. 12 through 14. In summary, it can be said that the treatment of HCM mice (αMHC^{719/+}) with the (R)-stereoisomer of a thiazolidinedione (using the example of pioglitazone) results in significantly lessened hypertrophy and fibrosis of the myocardium. The treatment of HCM mice (αMHC^{719/+}) with the (S)-stereoisomer of pioglitazone tends to result in lessened hypertrophy of myocardium (p=0.072), but not in a reduction of myocardial fibrosis.

### LITERATURE

1. Maron B J, Seidman C E, Ackerman M J, Towbin J A, Maron M S, Ommen S R, Nishimura R A, and Gersh B J. How should hypertrophic cardiomyopathy be classified ?: What's in a name? Dilemmas in nomenclature characterizing hypertrophic cardiomyopathy and left ventricular hypertrophy. Circ Cardiovasc Genet. 2009; 2: 81-5; discussion 86.
2. Maron B J. Hypertrophic cardiomyopathy: a systematic review. JAMA. 2002; 287: 1308-20.
3. Maron B J, McKenna W J, Danielson G K, Kappenberger L J, Kuhn H J, Seidman C E, Shah P M, Spencer W H, Spirito P, Ten Cate F J, Wigle E D, Vogel R A, Abrams J, Bates E R, Brodie B R, Danias P G, Gregoratos G, Hlatky M A, Hochman J S, Kaul S, Lichtenberg R C, Lindner J R, O'Rourke R A, Pohost G M, Schofield R S, Tracy C M, Winters W L, Klein W W, Priori S G, Alonso-Garcia A, Blomström-Lundqvist C, De Backer G, Deckers J, Flather M, Hradec J, Oto A, Parkhomenko A, Silber S, and Torbicki A. American College of Cardiology/European Society of Cardiology Clinical Expert Consensus Document on Hypertrophic Cardiomyopathy. A report of the American College of Cardiology Foundation Task Force on Clinical Expert Consensus Documents and the European Society of Cardiology Committee for Practice Guidelines. 2003; 24: 1965-1991.
4. Maron B J, Towbin J A, Thiene G, Antzelevitch C, Corrado D, Arnett D, Moss A J, Seidman C E, Young J B, American Heart A, Council on Clinical Cardiology H F, Transplantation C, Quality of C, Outcomes R, Functional G, Translational Biology Interdisciplinary Working G, Council on E and Prevention. Contemporary definitions and classification of the cardiomyopathies: an American Heart Association Scientific Statement from the Council on Clinical Cardiology, Heart Failure and Transplantation Committee; Quality of Care and Outcomes Research and Functional Genomics and Translational Biology Interdisciplinary Working Groups; and Council on Epidemiology and Prevention. Circulation. 2006; 113: 1807-16.
5. Gersh B J, Maron B J, Bonow R O, Dearani J A, Fifer M A, Link M S, Naidu S S, Nishimura R A, Ommen S R, Rakowski H, Seidman C E, Towbin J A, Udelson J E, and Yancy C W. 2011 ACCF/AHA guideline for the diagnosis and treatment of hypertrophic cardiomyopathy: executive summary: a report of the American College of Cardiology Foundation/American Heart Association Task Force on Practice Guidelines. J Am Coll Cardiol. 58: 2703-38.
6. Ommen S R, Mital S, Burke M A, Day S M, Deswal A, Elliott P, Evanovich L L, Hung J, Joglar J A, Kantor P, Kimmelstiel C, Kittleson M, Link M S, Maron M S, Martinez M W, Miyake C Y, Schaff H V, Semsarian C, and Sorajja P. 2020 AHA/ACC Guideline for the Diagnosis and Treatment of Patients With Hypertrophic Cardiomyopathy: A Report of the American College of Cardiology/American Heart Association Joint Committee on Clinical Practice Guidelines. Circulation. 2020; 142: e558-e631.
7. Semsarian C, Ahmad I, Giewat M, Georgakopoulos D, Schmitt J P, McConnell B K, Reiken S, Mende U, Marks A R, Kass D A, Seidman C E, and Seidman J G. The L-type calcium channel inhibitor diltiazem prevents cardiomyopathy in a mouse model. J Clin Invest. 2002; 109: 1013-20.
8. Westermann D, Knollmann B C, Steendijk P, Rutschow S, Riad A, Pauschinger M, Potter J D, Schultheiss H P, and Tschope C. Diltiazem treatment prevents diastolic heart failure in mice with familial hypertrophic cardiomyopathy. Eur J Heart Fail. 2006; 8: 115-21.
9. Teekakirikul P, Eminaga S, Toka O, Alcalai R, Wang L, Wakimoto H, Nayor M, Konno T, Gorham J M, Wolf C M, Kim J B, Schmitt J P, Molkentin J D, Norris R A, Tager A M, Hoffman S R, Markwald R R, Seidman C E, and Seidman J G. Cardiac fibrosis in mice with hypertrophic cardiomyopathy is mediated by non-myocyte proliferation and requires Tgf-beta. J Clin Invest. 120: 3520-9.
10. Ho C Y, Lakdawala N K, Cirino A L, Lipshultz S E, Sparks E, Abbasi S A, Kwong R Y, Antman E M, Semsarian C, Gonzalez A, Lopez B, Diez J, Orav E J, Colan S D, and Seidman C E. Diltiazem treatment for pre-clinical hypertrophic cardiomyopathy sarcomere mutation carriers: a pilot randomized trial to modify disease expression. JACC Heart Fail. 2015; 3: 180-8.
11. Shimada Y J, Passeri J J, Baggish A L, O'Callaghan C, Lowry P A, Yannekis G, Abbara S, Ghoshhajra B B, Rothman R D, Ho C Y, Januzzi J L, Seidman C E, and Fifer M A. Effects of losartan on left ventricular hypertrophy and fibrosis in patients with nonobstructive hypertrophic cardiomyopathy. JACC Heart Fail. 2013; 1: 480-7.
12. Axelsson A, Iversen K, Vejlstrup N, Ho C Y, Havndrup O, Kofoed K F, Norsk J, Jensen M, and Bundgaard H. Functional effects of losartan in hypertrophic cardiomyopathy-a randomised clinical trial. Heart. 2016; 102: 285-91.
13. Kawas R F, Anderson R L, Ingle S R B, Song Y, Sran A S, and Rodriguez H M. A small-molecule modulator of cardiac myosin acts on multiple stages of the myosin chemomechanical cycle. J Biol Chem. 2017; 292: 16571-16577.
14. Olivotto I, Oreziak A, Barriales-Villa R, Abraham T P, Masri A, Garcia-Pavia P, Saberi S, Lakdawala N K, Wheeler M T, Owens A, Kubanek M, Wojakowski W, Jensen M K, Gimeno-Blanes J, Afshar K, Myers J, Hegde S M, Solomon S D, Sehnert A J, Zhang D, Li W, Bhattacharya M, Edelberg J M, Waldman C B, Lester S J, Wang A, Ho C Y, Jacoby D, and investigators E-Hs. Mavacamten for treatment of symptomatic obstructive hypertrophic cardiomyopathy (EXPLORER-HCM): a randomised, double-blind, placebo-controlled, phase 3 trial. Lancet. 2020; 396: 759-769.
15. Berger J and Moller D E. The mechanisms of action of PPARs. Annu Rev Med. 2002; 53: 409-35.
16. Kliewer S A, Xu H E, Lambert M H, and Willson T M. Peroxisome proliferator-activated receptors: from genes to physiology. Recent Prog Horm Res. 2001; 56: 239-63.
17. Elbrecht A, Chen Y, Cullinan C A, Hayes N, Leibowitz M, Moller D E, and Berger J. Molecular cloning, expression and characterization of human peroxisome proliferator activated receptors gamma 1 and gamma 2. Biochem Biophys Res Commun. 1996; 224: 431-7.
18. Greene M E, Blumberg B, McBride O W, Yi H F, Kronquist K, Kwan K, Hsieh L, Greene G, and Nimer S D. Isolation of the human peroxisome proliferator activated receptor gamma cDNA: expression in hematopoietic cells and chromosomal mapping. Gene Expr. 1995; 4: 281-99.
19. Zieleniak A, Wojcik M, and Wozniak L A. Structure and physiological functions of the human peroxisome proliferator-activated receptor gamma. Arch Immunol Ther Exp (Warsz). 2008; 56: 331-45.
20. Michalik L, Auwerx J, Berger J P, Chatterjee V K, Glass C K, Gonzalez F J, Grimaldi P A, Kadowaki T, Lazar M A, O'Rahilly S, Palmer C N, Plutzky J, Reddy J K, Spiegelman B M, Staels B, and Wahli W. International Union of Pharmacology. LXI. Peroxisome proliferator-activated receptors. Pharmacological reviews. 2006; 58: 726-41.
21. Lehrke M and Lazar M A. The many faces of PPARgamma. Cell. 2005; 123: 993-9.
22. Yu S and Reddy J K. Transcription coactivators for peroxisome proliferator-activated receptors. Biochem Biophys Acta. 2007; 1771: 936-51.
23. Colca J R, Wyse B M, Sawada G, Jodelis K S, Connell C L, Fletcher-McGruder B L, Palazuk B J, and Diani A R. Ciglitazone, a hypoglycemic agent: early effects on the pancreatic islets of ob/ob mice. Metabolism. 1988; 37: 276-80.
24. Diani A R, Sawada G A, and Wyse B M. A review of the effects of ciglitazone on the pancreatic islets of obese, hyperglycemic mice. Prog Clin Biol Res. 1988; 265: 193-209.
25. Fujiwara T, Yoshioka S, Yoshioka T, Ushiyama I, and Horikoshi H. Characterization of new oral antidiabetic agent CS-045. Studies in KK and ob/ob mice and Zucker fatty rats. Diabetes. 1988; 37: 1549-58.
26. Sarhangi N, Sharifi F, Hashemian L, Hassani Doabsari M, Heshmatzad K, Rahbaran M, Jamaldini S H, Aghaei Meybodi H R, and Hasanzad M. PPARG (Pro12Ala) genetic variant and risk of T2DM: a systematic review and meta-analysis. Sci Rep. 2020; 10: 12764.
27. Rizos C V, Kei A, and Elisaf M S. The current role of thiazolidinediones in diabetes management. Arch Toxicol. 2016; 90: 1861-81.
28. Karak M, Bal N C, Bal C, and Sharon A. Targeting peroxisome proliferator-activated receptor gamma for generation of antidiabetic drug. Current diabetes reviews. 2013; 9: 275-85.
29. Derosa G and Maffioli P. Peroxisome proliferator-activated receptor-gamma (PPAR-gamma) agonists on glycemic control, lipid profile and cardiovascular risk. Curr Mol Pharmacol. 2012; 5: 272-81.
30. Spiegelman B M. Peroxisome proliferator-activated receptor gamma: A key regulator of adipogenesis and systemic insulin sensitivity. Eur J Med Res. 1997; 2: 457-64.
31. Kokeny G, Calvier L, Legchenko E, Chouvarine P, Mozes M M, and Hansmann G. PPARgamma is a gatekeeper for extracellular matrix and vascular cell homeostasis: beneficial role in pulmonary hypertension and renal/cardiac/pulmonary fibrosis. Curr Opin Nephrol Hypertens. 2020; 29: 171-179.
32. Hansmann G, Calvier L, Risbano M G, and Chan S Y. Activation of the Metabolic Master Regulator PPARgamma: A Potential Pioneering Therapy for Pulmonary Arterial Hypertension. American journal of respiratory cell and molecular biology. 2020; 62: 143-156.
33. DeWitt S, Czarnik A W, and Jacques V. Deuterium-Enabled Chiral Switching (DECS) Yields Chirally Pure Drugs from Chemically Interconverting Racemates. ACS Med Chem Lett. 2020; 11: 1789-1792.
34. Divakaruni A S, Wiley SE, Rogers G W, Andreyev A Y, Petrosyan S, Loviscach M, Wall E A, Yadava N, Heuck A P, Ferrick D A, Henry R R, McDonald W G, Colca J R, Simon M I, Ciaraldi T P, and Murphy A N. Thiazolidinediones are acute, specific inhibitors of the mitochondrial pyruvate carrier. Proc Natl Acad Sci USA. 2013; 110: 5422-7.
35. Jamali B, Bjornsdottir I, Nordfang O, and Hansen S H. Investigation of racemisation of the enantiomers of glitazone drug compounds at different pH using chiral HPLC and chiral CE. J Pharm Biomed Anal. 2008; 46: 82-7.
36. Geisterfer-Lowrance A A, Christe M, Conner D A, Ingwall J S, Schoen F J, Seidman C E, and Seidman J G. A mouse model of familial hypertrophic cardiomyopathy. Science. 1996; 272: 731-4.
37. Wolf C M, Moskowitz I P, Arno S, Branco D M, Semsarian C, Bernstein S A, Peterson M, Maida M, Morley G E, Fishman G, Berul C I, Seidman C E, and Seidman J G. Somatic events modify hypertrophic cardiomyopathy pathology and link hypertrophy to arrhythmia. Proc Natl Acad Sci USA. 2005; 102: 18123-8.
38. Teekakirikul P, Eminaga S, Toka O, Alcalai R, Wang L, Wakimoto H, Nayor M, Konno T, Gorham J M, Wolf C M, Kim J B, Schmitt J P, Molkentin J D, Norris R A, Tager A M, Hoffman S R, Markwald R R, Seidman C E, and Seidman J G. Cardiac fibrosis in mice with hypertrophic cardiomyopathy is mediated by non-myocyte proliferation and requires Tgf-beta. J Clin Invest. 2010; 120: 3520-9.
39. Fatkin D, McConnell B K, Mudd J O, Semsarian C, Moskowitz I G, Schoen F J, Giewat M, Seidman C E, and Seidman J G. An abnormal Ca(2+) response in mutant sarcomere protein-mediated familial hypertrophic cardiomyopathy. J Clin Invest. 2000; 106: 1351-9.
40. Wolf C M, Arad M, Ahmad F, Sanbe A, Bernstein S A, Toka O, Konno T, Morley G, Robbins J, Seidman J G, Seidman C E, and Berul C I. Reversibility of PRKAG2 glycogen-storage cardiomyopathy and electrophysiological manifestations. Circulation. 2008; 117: 144-54.
41. Wolf C M, Wang L, Alcalai R, Pizard A, Burgon P G, Ahmad F, Sherwood M, Branco D M, Wakimoto H, Fishman G I, See V, Stewart C L, Conner D A, Berul C I, Seidman C E, and Seidman J G. Lamin A/C haploinsufficiency causes dilated cardiomyopathy and apoptosis-triggered cardiac conduction system disease. J Mol Cell Cardiol. 2008; 44: 293-303.
42. Semsarian C, Ahmad I, Giewat M, Georgakopoulos D, Schmitt J P, McConnell B K, Reiken S, Mende U, Marks A R, Kass D A, Seidman C E, and Seidman J G. The L-type calcium channel inhibitor diltiazem prevents cardiomyopathy in a mouse model. Journal of Clinical Investigation. 2002; 109: 1013-1020.
43. Arad M, Moskowitz I P, Patel V V, Ahmad F, Perez-Atayde A R, Sawyer D B, Walter M, Li G H, Burgon P G, Maguire C T, Stapleton D, Schmitt J P, Guo X X, Pizard A, Kupershmidt S, Roden D M, Berul C I, Seidman C E, and Seidman J G. Transgenic mice overexpressing mutant PRKAG2 define the cause of Wolff-Parkinson-White syndrome in glycogen storage cardiomyopathy. Circulation. 2003; 107: 2850-6.
44. Jahn W. The association of actin and myosin in the presence of gamma-amido-ATP proceeds mainly via a complex with myosin in the closed conformation. Biochemistry. 2007; 46: 9654-64.
45. LaConte L E W, Srivastava S, and Mukherjee K. Probing Protein Kinase-ATP Interactions Using a Fluorescent ATP Analog. Methods Mol Biol. 2017; 1647: 171-183.
46. Diebold I, Petry A, Hess J, and Gorlach A. The NADPH oxidase subunit NOX4 is a new target gene of the hypoxia-inducible factor-1. Molecular biology of the cell. 2010; 21: 2087-96.
47. Rzymski T, Petry A, Kracun D, Riess F, Pike L, Harris A L, and Gorlach A. The unfolded protein response controls induction and activation of ADAM17/TACE by severe hypoxia and ER stress. Oncogene. 2012; 31: 3621-34.
48. Hewing B, Ludwig A, Dan C, Potzsch M, Hannemann C, Petry A, Lauer D, Gorlach A, Kaschina E, Muller D N, Baumann G, Stangl V, Stangl K, and Wilck N. Immunoproteasome subunit ss5i/LMP7-deficiency in atherosclerosis. Scientific reports. 2017; 7: 13342.
49. Pfaffl M W. A new mathematical model for relative quantification in real-time RT-PCR. Nucleic Acids Research. 2001; Vol. 29.
50. Michael W. Pfaffl GHaLD. Relative expression software tool (REST©) for group-wise comparison and statistical analysis of relative expression results in real-time PCR. Nucleic Acids Research. 2002; 2002.
51. Haack T B, Kopajtich R, Freisinger P, Wieland T, Rorbach J, Nicholls T J, Baruffini E, Walther A, Danhauser K, Zimmermann F A, Husain R A, Schum J, Mundy H, Ferrero I, Strom T M, Meitinger T, Taylor R W, Minczuk M, Mayr J A, and Prokisch H. ELAC2 mutations cause a mitochondrial RNA processing defect associated with hypertrophic cardiomyopathy. American journal of human genetics. 2013; 93: 211-23.
52. Dobin A, Davis C A, Schlesinger F, Drenkow J, Zaleski C, Jha S, Batut P, Chaisson M, and Gingeras T R. STAR: ultrafast universal RNA-seq aligner. Bioinformatics. 2013; 29: 15-21.
53. Anders S, Pyl P T, and Huber W. HTSeq—a Python framework to work with high-throughput sequencing data. Bioinformatics. 2015; 31: 166-9.
54. Love M I, Huber W, and Anders S. Moderated estimation of fold change and dispersion for RNA-seq data with DESeq2. Genome Biol. 2014; 15: 550.
55. Lee K S, Kim S R, Park S J, Park H S, Min K H, Jin S M, Lee M K, Kim U H, and Lee Y C. Peroxisome proliferator activated receptor-gamma modulates reactive oxygen species generation and activation of nuclear factor-kappaB and hypoxia-inducible factor 1alpha in allergic airway disease of mice. J Allergy Clin Immunol. 2006; 118: 120-7.
56. Hinson J T, Chopra A, Lowe A, Sheng C C, Gupta R M, Kuppusamy R, O'Sullivan J, Rowe G, Wakimoto H, Gorham J, Burke M A, Zhang K, Musunuru K, Gerszten R E, Wu S M, Chen C S, Seidman J G, and Seidman C E. Integrative Analysis of PRKAG2 Cardiomyopathy iPS and Microtissue Models Identifies AMPK as a Regulator of Metabolism, Survival, and Fibrosis. Cell Rep. 2017; 19: 2410.

The literature cited above is incorporated in its entirety into the disclosure of the present application.

1. PPARγ agonist and/or MPC inhibitor for use in a method for preventing or treating a primary, congenital cardiomyopathy.

2. PPARγ agonist or MPC inhibitor for use in a method according to claim 1, wherein the primary, congenital cardiomyopathy is selected from the group consisting of hypertrophic cardiomyopathy (HCM), dilated cardiomyopathy (DCM), restrictive HCM, a cardiomyopathy in structural congenital heart defects and in RASopathies—in particular, diseases from the Noonan syndrome spectrum.

3. PPARγ agonist or MPC inhibitor for use in a method according to claim 1 or claim 2, wherein the primary, congenital cardiomyopathy is selected from the group consisting of hypertrophic cardiomyopathy (HCM), a cardiomyopathy in structural, congenital heart defects and in RASopathies—in particular, diseases from the Noonan syndrome spectrum.

4. PPARγ agonist or MPC inhibitor for use in a method according to one of claims 1 through 3, wherein the primary cardiomyopathy is a hypertrophic cardiomyopathy.

5. PPARγ agonist or MPC inhibitor for use in a method according to one of claims 1 through 4, wherein the PPARγ agonist or MPC inhibitor is a compound from the group of thiazolidine-2,4-diones.

6. PPARγ agonist or MPC inhibitor for use in a method according to one of claims 1 through 5, wherein the PPARγ agonist or MPC inhibitor is a thiazolidine-2,4-dione substituted at the C5 ring atom.

7. PPARγ agonist or MPC inhibitor for use in a method according to one of claims 1 through 6, wherein the PPARγ agonist or MPC inhibitor has a substituent at the C5 ring atom, which substituent has two (hetero)aromatic ring systems connected to one another via a linker structure.

8. PPARγ agonist or MPC inhibitor for use in a method according to one of claims 1 through 7, wherein the PPARγ agonist or MPC inhibitor is a glitazone—in particular, selected from the group consisting of ciglitazone, balaglitazone, darglitazone, englitazone, netoglitazone, pioglitazone, rivoglitazone, rosiglitazone, GQ-16, and troglitazone—wherein the PPARγ agonist or MPC inhibitor is preferably pioglitazone or rosiglitazone.

9. PPARγ agonist or MPC inhibitor for use in a method according to one of claims 1 through 8, wherein the PPARγ agonist or MPC inhibitor is a salt of an inorganic or organic acid, and in particular a salt of hydrochloric acid or of maleic acid.

10. PPARγ agonist or MPC inhibitor for use in a method according to one of claims 1 through 9, wherein the PPARγ agonist or MPC inhibitor is an (S)-enantiomer or an (R)-enantiomer, and preferably an (S)-enantiomer or (R)-enantiomer deuterated at the chiral center.

11. PPARγ agonist or MPC inhibitor for use in a method according to one of claims 1 through 9, wherein the PPARγ agonist or MPC inhibitor is an (R)-enantiomer, and preferably an (R)-enantiomer deuterated at the chiral center.

12. Pharmaceutical composition, containing a PPARγ agonist or MPC inhibitor according to one of claims 1 through 11 and optionally a pharmaceutical carrier, for use in a method for preventing or treating a primary, congenital cardiomyopathy.

13. Pharmaceutical composition for use in a method according to claim 12, wherein the primary, congenital cardiomyopathy is selected from the group consisting of hypertrophic cardiomyopathy (HCM), dilated cardiomyopathy (DCM), restrictive HCM, a cardiomyopathy in structural congenital heart defects and RASopathies—in particular, diseases from the Noonan syndrome spectrum, or in particular hypertrophic cardiomyopathy (HCM).

14. Pharmaceutical composition for use in a method according to claim 12 or 13, wherein the primary, congenital cardiomyopathy is selected from the group consisting of hypertrophic cardiomyopathy (HCM), a cardiomyopathy in structural, congenital heart defects and RASopathies—in particular, diseases from the Noonan syndrome spectrum, or in particular hypertrophic cardiomyopathy (HCM).

15. Pharmaceutical composition for use in a method according to claim 12 or 13, wherein the composition contains an (S)-enantiomer or (R)-enantiomer of a glitazone—preferably with a deuterium atom at the chiral center—in enriched or in pure form, or a racemate of the (S)-enantiomer and (R)-enantiomer of a glitazone.

16. Pharmaceutical composition for use in a method according to claim 15, wherein the composition contains an (R)-enantiomer of a glitazone—preferably with a deuterium atom at the chiral center—in enriched or pure form.

17. Pharmaceutical composition for use in a method according to one of claims 12 through 16, wherein the pharmaceutical composition is a therapeutic composition to be administered orally, and in particular a coated tablet.

18. PPARγ agonist or MPC inhibitor for use in a method according to one of claims 1 through 11, or the pharmaceutical composition for use in a method according to one of claims 12 through 17, wherein the PPARγ agonist or MPC inhibitor is administered in a daily dose of 1 to 30 mg, and preferably 1 to 15 mg.

19. PPARγ agonist or MPC inhibitor for use in a method according to one of claims 1 through 11, or the pharmaceutical composition for use in a method according to one of claims 12 through 18, wherein the method comprises a combination therapy with an active agent selected from the group consisting of a beta blocker, an angiotensin-converting enzyme (ACE) inhibitor, an angiotensin I receptor blocker, an aldosterone antagonist, a calcium antagonist, an anti-arrhythmic drug, and a myosin modulator.
